(19) United States
(12) Patent Application Publication
(10) Pub. No.: US 2024/0081561 A1
(43) Pub. Date: Mar. 14, 2024

(54) Heated and Cooled Corner Pillow Device

(71) Applicants: David Arnett, Bolivar, MO (US); Katherine Arnett, Bolivar, MO (US)

(72) Inventors: David Arnett, Bolivar, MO (US); Katherine Arnett, Bolivar, MO (US)

(21) Appl. No.: 18/447,688

(22) Filed: Aug. 10, 2023

Related U.S. Application Data
(60) Provisional application No. 63/404,727, filed on Sep. 8, 2022.

(51) Int. Cl. A47G 9/10; A61F 7/08; A61F 7/10

(52) U.S. Cl. CPC A47G 9/1036; A61F 7/08; A61F 7/10; A47C 7/425

(57) ABSTRACT

A heated and cooled corner pillow device is provided. The device is comprised of a body that has a first pillow, a second pillow, a third pillow, and a stopper. The pillows each have a pocket to accommodate an insert of a heating or cooling pad. Additionally, the body is angled to fit in the corner of a couch. The stopper is tucked in between the couch cushions to secure the device in place during use so it does not move.

### CROSS-REFERENCE TO RELATED APPLICATION

The present application claims priority to, and the benefit of, U.S. Provisional Application No. 63/404,727, which was filed on Sep. 8, 2022, and is incorporated herein by reference in its entirety.

### FIELD OF THE INVENTION

The present invention relates generally to the field of pillows. More specifically, the present invention relates to a heated and cooled corner pillow device comprised of at least one cushioned pillow that can accept at least one heating or cooling pad. Accordingly, the present disclosure makes specific reference thereto. Nonetheless, it is to be appreciated that aspects of the present invention are also equally applicable to other like applications, devices, and methods of manufacture.

### BACKGROUND

When sitting on a couch or sofa, individuals may use heating and cooling pads to enhance their comfort, alleviate pain, promote circulation, etc. However, said pads are difficult to maintain in a fixed position while sitting or lying on the couch. Furthermore, pillows can be challenging to keep in the correct spot to optimize comfort when laying, sitting, relaxing, etc. on a couch. Additionally, traditional pillows do not reliably provide support when utilizing heating or cooling pads. Moreover, traditional pillows can become misshapen, deformed, etc., when laying, sitting, relaxing, etc., for a prolonged period of time, which results in an uncomfortable seating experience.

Therefore, there exists a long-felt need in the art for a device that secures heating and cooling pads in place for optimal comfort, pain alleviation, circulation promotion, etc. More specifically, there exists a long-felt need in the art for a heated and cooled corner pillow device that can be secured on a couch during use. Moreover, there exists a long-felt need in the art for a heated or cooled corner pillow device that can be secured to a couch back or corner with at least one stopper attachment piece and at least one slot for receiving at least one heating, cooling, or another pad.

The subject matter disclosed and claimed herein, in one embodiment thereof, comprises a heated and cooled corner pillow device. The device is comprised of at least one pillow with at least one slot positioned on the front of the pillow. The slot can receive heating, cooling, or another pad known in the art. Additionally, the pillow connects to at least one stopper that fits in between couch cushions to secure the device in place during use. While attached to a couch, the device provides support for sitting or lying on the couch and holding at least one heating, cooling, or another pad known in the art.

In this manner, the heated and cooled corner pillow device of the present invention accomplishes all the foregoing objectives and provides a device that secures at least one heating, cooling, or other pad in place to provide comfort, alleviate pain, promote circulation, etc., while sitting or lying. More specifically, the heated and cooled corner pillow device can be fixed to a couch's back, corner, or armrest with at least one stopper positioned between the couch cushions or seams to secure the device during use to provide a better sitting experience than existing pillows by minimizing pillow movement.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a heated and cooled corner pillow device comprised of the first pillow, the second pillow, the third pillow, and a stopper. The first pillow is attached to the second pillow via a seam. Additionally, the second pillow is attached to the third pillow via a seam. In the preferred embodiment of the device, the body forms an angle between 90 and 180 degrees.

During use, a user can sit, lay, relax, etc. against the first pillow. The first pillow is generally a triangular shape, in the preferred embodiment, with a bottom surface that supports the device. Additionally, the first pillow is comprised of at least one cover with at least one pocket. The pocket receives at least one heating or cooling pad. A user can also sit, lay, relax, etc. against the second pillow. The second pillow is generally a triangular shape, in the preferred embodiment, with a bottom surface that supports the device. Additionally, the second pillow is comprised of at least one cover with at least one pocket. The pocket receives at least one heating or cooling pad. Furthermore, a user can sit, lay, relax, etc. against the third pillow. The third pillow is generally a triangular shape, in the preferred embodiment, with a bottom surface that supports the device. Additionally, the third pillow is comprised of at least one cover with at least one pocket. The pocket receives at least one heating or cooling pad.

While being used, the device is preferably positioned in the corner of a couch. The stopper secures the device to the couch once the stopper is tucked in between a sitting cushion and a seam of the couch. In the preferred embodiment, the stopper is triangular in shape. The stopper prevents the device from shifting in position during the use.

The present invention is also comprised of a first method of using the device. First, a device is provided comprised of a body comprised of a first pillow, a second pillow, a third pillow, and a stopper. A user can then attach the body to a couch via the stopper by securing the stopper between a sitting cushion and a seam of the couch. Next, a user can place at least one heating pad in at least one of the pockets of the first pillow, second pillow, or third pillow. Then, the user sits, lies, relaxes, etc. against the body of the device.

The present invention is also comprised of a second method of using the device. First, a device is provided comprised of a body comprised of a first pillow, the second pillow, the third pillow, and a stopper. A user can then attach the body to a couch via the stopper by securing the stopper between a sitting cushion and a seam of the couch. Next, a user can place at least one cooling pad in at least one of the pockets of the first pillow, the second pillow, or the third pillow. Then, the user sits, lies, relaxes, etc. against the body of the device.

Accordingly, the heated and cooled corner pillow device of the present invention is particularly advantageous as it provides a device that secures at least one heating, cooling, or another pad in place while sitting or lying on the couch. The optimally secured heating, cooling, or other pads provide the user comfort, alleviate pain, promote circulation, etc., without the inconvenience of the pads sliding around while sitting or lying on the couch. Additionally, the heated and cooled corner pillow device can be secured to the couch's back, corner, armrest, or other area of the couch, ensuring optimal seating, or lying comfort. More specifically, the heated and cooled corner pillow device is comprised of at least one stopper that can be wedged between couch cushions or seams to ensure the device does not migrate during use. In this manner, the heated and cooled corner pillow device overcomes the limitations of existing pillows known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a heated and cooled corner pillow device of the present invention in accordance with the disclosed architecture;

FIG. 2 illustrates a bottom view of one potential embodiment of a heated and cooled corner pillow device of the present invention in accordance with the disclosed architecture;

FIG. 3 illustrates a perspective view of one potential embodiment of a heated and cooled corner pillow device of the present invention while attached to a couch in accordance with the disclosed architecture;

FIG. 4 illustrates a flowchart of a first method of using one potential embodiment of a heated and cooled corner pillow device of the present invention in accordance with the disclosed architecture; and

FIG. 5 illustrates a flowchart of a first method of using one potential embodiment of a heated and cooled corner pillow device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that reliably secures heating and cooling pads in place for optimal comfort, pain alleviation, circulation promotion, etc., while sitting, lying down, etc. More specifically, there exists a long-felt need in the art for a heated and cooled corner pillow device that can be secured to a couch back or corner with at least one stopper attachment piece. Moreover, there exists a long-felt need in the art for a heated or cooled corner pillow device that can be secured to a couch back or corner with at least one stopper attachment piece and at least one slot for at least one heating, cooling, or another pad known in the art.

The present invention, in one exemplary embodiment, is comprised of a heated and cooled corner pillow device. The device is comprised of a first pillow, a second pillow, a third pillow, and a stopper. Together, the pillows and the stopper comprise the body of the device. Each pillow is attached to one another via a seam. The body is designed to be angled between 90 and 180 degrees.

The first pillow of the device is a triangular shape that the user can sit, lay, relax, etc., up against. The first pillow has a bottom surface that supports the device on the sitting cushion of the couch. Furthermore, the first pillow is comprised of at least one cover comprised of a pocket. The pocket of the first pillow receives at least one heated, cooled, or other pad insert.

The second pillow of the device is a triangular shape that the user can sit, lay, relax, etc., up against. The second pillow has a bottom surface that supports the device on the sitting cushion of the couch. Furthermore, the second pillow is comprised of at least one cover comprised of a pocket. The pocket of the second pillow receives at least one heated, cooled, or other pad insert.

The third pillow of the device is a triangular shape that the user can sit, lay, relax, etc., up against. The third pillow has a bottom surface that supports the device on the sitting cushion of the couch. Furthermore, the third pillow is comprised of at least one cover comprised of a pocket. The pocket of the third pillow receives at least one heated, cooled, or other pad insert.

The device is positioned for use in the corner of the couch. The device is secured in this position on the couch by the stopper. The stopper is of a triangular shape and is tucked between the sitting cushion and the seam of the couch. Additionally, the stopper prevents the device from moving during use.

The present invention is also comprised of a first method of using the device. First, the device is comprised of the first pillow, the second pillow, the third pillow, and the stopper that comprise the body. Then, a user attaches the device to the couch via the stopper by securing the stopper between the sitting cushion and the seam of the couch. Next, a user places at least one heating pad in at least one of the pockets of the first pillow, second pillow, or third pillow. Then, the user sits, lies, relaxes, etc., on the body of the device.

The present invention is also comprised of a second method of using the device. First, the device is comprised of the first pillow, the second pillow, the third pillow, and the stopper that comprise the body. Then, a user attaches the device to the couch via the stopper by securing the stopper between the sitting cushion and the seam of the couch. Next, a user places at least one cooling pad in at least one of the pockets of the first pillow, second pillow, or third pillow. Then, the user sits, lies, relaxes, etc. on the body of the device.

Accordingly, the heated and cooled corner pillow device of the present invention is particularly advantageous as it provides a device that secures at least one heating, cooling, or another pad in place while sitting or lying on the couch. The secured heating, cooling, or other pads provide the user comfort, alleviate pain, promote circulation, etc., without the inconvenience of the pads sliding around while sitting or lying on the couch. Furthermore, the heated and cooled corner pillow device can be secured to the couch back, corner, armrest, or other areas of the couch, ensuring optimal seating, or lying comfort. More specifically, the heated and cooled corner pillow device is comprised of at least one stopper that can be wedged between couch cushions or seams to ensure the device does not shift position/move during use. In this manner, the heated and cooled corner pillow device overcomes the limitations of existing pillows known in the art.

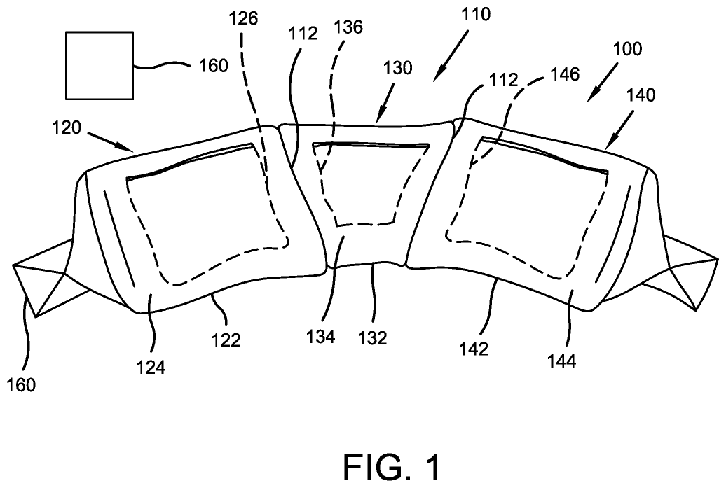

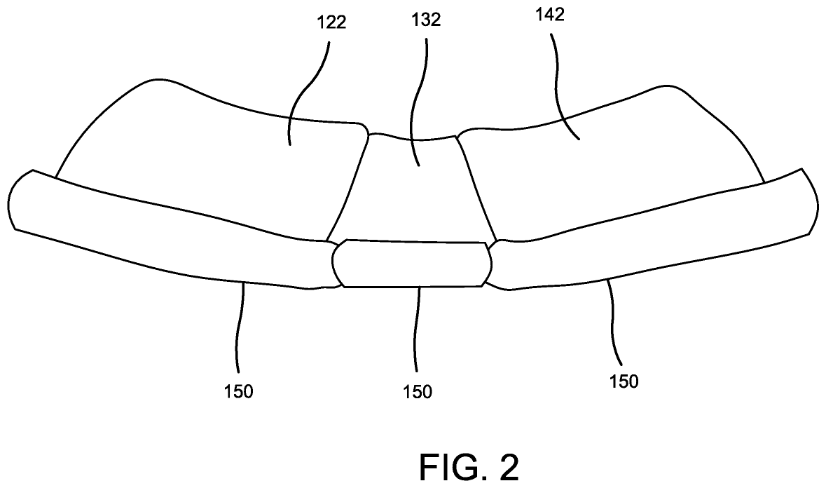

Referring initially to the drawings, FIG. 1 illustrates a perspective view of the device 100. The device 100 is comprised of a body 110 comprised of at least a first pillow 120 and a second pillow 130, an optional third pillow 140, and at least one stopper 150. The body 110 may be made from any fabric known in the art, such as but not limited to cotton, silk, linen, wool, velvet, etc. The first pillow 120 is attached to the second pillow 130 via at least one seam 112. The second pillow 130 is attached to the third pillow 140 via at least one second seam 112. In the preferred embodiment, the first pillow 120, the second pillow 130, the third pillow 140 form an angle between 90 and 180 degrees, as seen in FIG. 2. In another embodiment, the body 110 may be angled less than 90 degrees or more than 180 degrees. The angle of the body 110 supports a user's body while sitting or lying against the body 110.

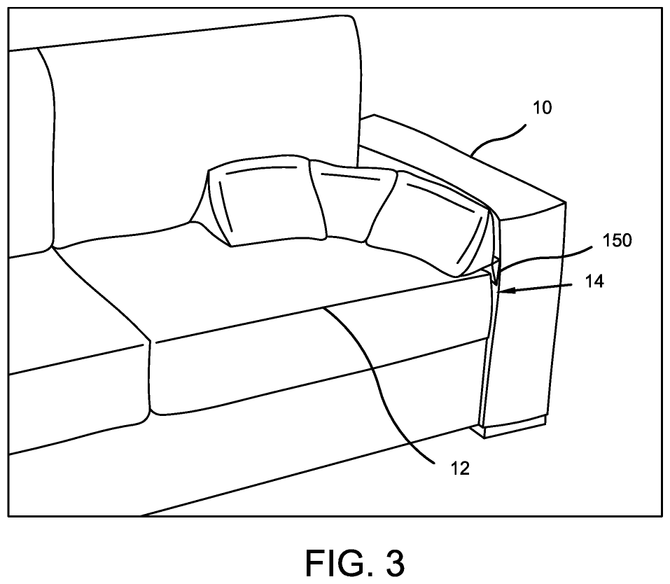

The first pillow 120 is a section of the device 100 that the user can sit, lay, or relax against. The first pillow 120 may be made from any material known in the art, such as but not limited to foam, memory foam, cotton, feathers, latex, down, etc. Additionally, the first pillow 120 is preferably triangular in shape with a flat bottom surface 122, although the first pillow 120 may be any shape in different embodiments to provide support to the user's body during use. The bottom surface 122 rests on the sitting cushion 12, best seen in FIG. 3, to provide support and comfort while the user is sitting, lying, or relaxing on the device 100. Furthermore, the first pillow 120 is comprised of at least one cover 124 comprised of at least one pocket 126. In the preferred embodiment, the cover 124 is made of the same material as the fabric of the body 110. In another embodiment, the cover 124 may be made from another material known in the art, such as but not limited to cotton, silk, linen, wool, velvet, etc. The pocket 126 receives at least one insert 160. The insert 160 may be any type of heating or cooling pad known in the art, such as but not limited to an instant ice pack, gelled ice pack, a refillable ice pack, an electric heating pad, an infrared heating pad, a chemical heating pad, a gel heating pad, etc.

The second pillow 130 is a section of the device 100 that the user can sit, lay, or relax against. The second pillow 130 may be made from any material known in the art, such as but not limited to foam, memory foam, cotton, feathers, latex, down, etc. Additionally, the second pillow 130 is preferably triangular in shape with a flat bottom surface 132. Although, the second pillow 130 may be any shape in different embodiments to provide support to the user's body during use. The bottom surface 132 rests on the sitting cushion 12, best seen in FIG. 3, to provide support and comfort while the user is sitting, lying, or relaxing on the device 100. Furthermore, the second pillow 130 is comprised of at least one cover 134 comprised of at least one pocket 136. In the preferred embodiment, the cover 134 is made of the same material as the fabric of the body 110. In another embodiment, the cover 134 may be made from another material known in the art, such as but not limited to cotton, silk, linen, wool, velvet, etc. The pocket 136 receives at least one insert 160. The insert 160 may be any type of heating or cooling pad known in the art, such as but not limited to an instant ice pack, gelled ice pack, a refillable ice pack, an electric heating pad, an infrared heating pad, a chemical heating pad, a gel heating pad, etc.

The third pillow 140 is a section of the device 100 that the user can sit, lay, or relax against. The third pillow 140 may be made from any material known in the art, such as but not limited to foam, memory foam, cotton, feathers, latex, down, etc. Additionally, the third pillow 140 is preferably triangular in shape with a flat bottom surface 142, although the third pillow 140 may be any shape in different embodiments to provide support to the user's body during use. The bottom surface 142 rests on the sitting cushion 12, best seen in FIG. 3, to provide support and comfort while the user is sitting, lying, or relaxing on the device 100. Furthermore, the third pillow 140 is comprised of at least one cover 144 comprised of at least one pocket 146. In the preferred embodiment, the cover 144 is made of the same material as the fabric of the body 110. In another embodiment, the cover 144 may be made from another material known in the art, such as but not limited to cotton, silk, linen, wool, velvet, etc. The pocket 146 receives at least one insert 160. The insert 160 may be any type of heating or cooling pad known in the art, such as but not limited to an instant ice pack, gelled ice pack, a refillable ice pack, an electric heating pad, an infrared heating pad, a chemical heating pad, a gel heating pad, etc.

Referring to FIG. 3, the device 100 is preferably positioned in a corner of a couch 10 during use. However, the device 100 may be positioned on any location of a couch in different embodiments. The device 100 is removably secured to the couch 10 by at least one stopper 150. The stopper 150 is tucked between a sitting cushion 12 and a seam 14 of the couch to secure the device 100 so it does not shift/move during use. In the preferred embodiment of the device 100, the stopper 150 is the general shape of a triangle. However, in another embodiment, the stopper 150 can be any other shape known in the art, such as but not limited to a circle, cylinder, rectangle, etc.

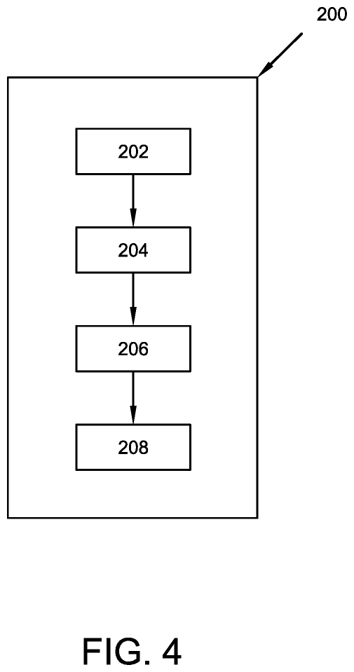

The present invention is also comprised of a first method of using 200 the device 100, as seen in FIG. 4. First, a device 100 is provided comprised of a body 110 comprised of a first pillow 120, a second pillow 130, a third pillow 140, and a stopper 150 [Step 202]. Then, a user can attach the body 110 to a couch 10 via placing the stopper 150 between a sitting cushion 12 and a seam 14 of the couch [Step 204]. Next, a user can insert at least one heating pad insert 160 into the pocket 126 of the first pillow 120, the pocket 136 of the second pillow 130, or the pocket 146 of the third pillow 140 [Step 206]. Then, a user can sit, lie, relax, etc., against/on the body 110 of the device 100 [Step 208].

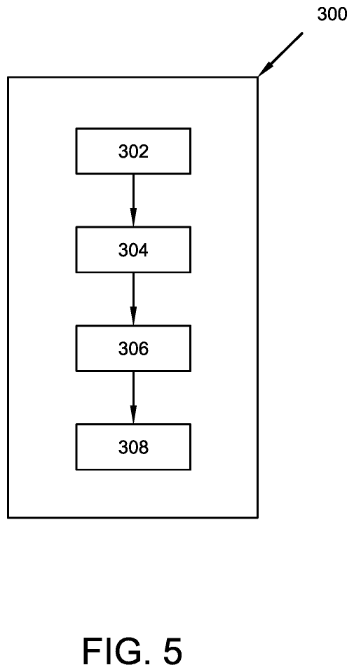

The present invention is also comprised of a second method of using 300 the device 100, as seen in FIG. 5. First, a device 100 is provided comprised of a body 110 comprised of a first pillow 120, a second pillow 130, a third pillow 140, and a stopper 150 [Step 302]. Then, a user can attach the body 110 to a couch 10 via placing the stopper 150 between a sitting cushion 12 and a seam 14 of the couch [Step 304]. Next, a user can insert at least one heating pad insert 160 into the pocket 126 of the first pillow 120, the pocket 136 of the second pillow 130, or the pocket 146 of the third pillow 140 [Step 306]. Then, a user can sit, lie, relax, etc. against/on the body 110 of the device 100 [Step 308].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “heated and cooled corner pillow device” and “device” are interchangeable and refer to the heated and cooled corner pillow device 100 of the present invention.

Notwithstanding the foregoing, the heated and cooled corner pillow device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the heated and cooled corner pillow device 100 as shown in the FIGS. are for illustrative purposes only, and that many other sizes and shapes of the heated and cooled corner pillow device 100 are well within the scope of the present disclosure. Although the dimensions of the heated and cooled corner pillow device 100 are important design parameters for user convenience, the heated and cooled corner pillow device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A heated and cooled corner pillow device comprising:
a first pillow comprised of a first pocket;
a second pillow attached to the first pillow, the second pillow comprised of a second pocket;
a stopper attached to the first pillow and the second pillow; and
a pad.

2. The heated and cooled corner pillow device of claim 1, wherein the pad is comprised of a heating pad.

3. The heated and cooled corner pillow device of claim 2, wherein the heating pad is comprised of an electric heating pad, an infrared heating pad, a chemical heating pad, or a gel heating pad.

4. The heated and cooled corner pillow device of claim 1, wherein the pad is comprised of a cooling pad.

5. The heated and cooled corner pillow device of claim 4, wherein the cooling pad is comprised of an instant ice pack, a gelled ice pack, or a refillable ice pack.

6. The heated and cooled corner pillow device of claim 1, wherein the stopper is comprised of a triangular shape.

7. The heated and cooled corner pillow device of claim 1, wherein the first pillow and the second pillow are each comprised of a triangular shape.

8. The heated and cooled corner pillow device of claim 1, wherein the first pillow and the second pillow are each comprised of a flat bottom surface.

9. The heated and cooled corner pillow device of claim 1, wherein the first pillow and the second pillow are each comprised of a foam, a memory foam, a cotton, a feather, a latex, or a down material.

10. A heated and cooled corner pillow device comprising:
a first pillow comprised of a pocket;
a second pillow attached to the first pillow, the second pillow comprised of a second pocket;
a third pillow attached to the second pillow, the third pillow comprised of a third pocket;
a stopper attached to the first pillow, the second pillow, and the third pillow; and
a pad.

11. The heated and cooled corner pillow device of claim 10, wherein the pad is comprised of a heating pad.

12. The heated and cooled corner pillow device of claim 11, wherein the heating pad is comprised of an electric heating pad, an infrared heating pad, a chemical heating pad, or a gel heating pad.

13. The heated and cooled corner pillow device of claim 10, wherein the pad is comprised of a cooling pad.

14. The heated and cooled corner pillow device of claim 13, wherein the cooling pad is comprised of an instant ice pack, a gelled ice pack, or a refillable ice pack.

15. The heated and cooled corner pillow device of claim 10, wherein the stopper is comprised of a triangular shape.

16. The heated and cooled corner pillow device of claim 10, wherein the first pillow and the second pillow, and the third pillow are each comprised of a triangular shape.

17. The heated and cooled corner pillow device of claim 10, wherein the first pillow, the second pillow, and the third pillow form an angle between 90 and 180 degrees.

18. The heated and cooled corner pillow device of claim 10, wherein the first pillow, the second pillow, and the third pillow are each comprised of a flat bottom surface.

19. The heated and cooled corner pillow device of claim 10, wherein the first pillow, the second pillow, and the third pillow are each comprised of a foam, a memory foam, a cotton, a feather, a latex, or a down material.

20. A method of using a heated and cooled corner pillow device, the method comprising the following steps:
providing a heated and cooled corner pillow device comprised of a device comprised of a first pillow, a second pillow, a third pillow, and a stopper;
attaching the body to a couch via placing the stopper between a sitting cushion and a seam of the couch;
inserting a pad insert into a pocket of the first pillow, a pocket of the second pillow, or a pocket of the third pillow; and
sitting or lying against the first pillow, the second pillow, or the third pillow.
